(19) United States
(12) Patent Application Publication
(10) Pub. No.: US 2013/0112196 A1
(43) Pub. Date: May 9, 2013

(54) Disposable protective device for suction tube

(71) Applicant: Tien-Hsiung Ku, Changhua (TW)

(72) Inventor: Tien-Hsiung Ku, Changhua (TW)

(73) Assignee: Changhua Christian Hospital

(21) Appl. No.: 13/659,900

(22) Filed: Oct. 24, 2012

(30) Foreign Application Priority Data
Oct. 27, 2011 (TW) 100220182

(51) Int. Cl. A61M 1/00; A61M 16/04

(52) U.S. Cl. 128/202.16

(57) ABSTRACT

The disposable protective device for suction tube includes an encapsulating bag unit and a connective tubular unit. The encapsulating bag unit has a protruding circular portion. The encapsulating bag unit has an interior space which may house a suction tube. The encapsulating bag unit has a first opening and a second opening at its two ends, with the diameter of the second opening larger than or equal to that of the first opening. The connective tubular unit has a first connector, a second connector and a third connector. The first connector may connect with the second opening. A cover may cover the first connector. The second connector may connect with a ventilator. The third connector is provided at the bottom part of the connective tubular unit and may connect with a tracheostomy tube or a tube inside the trachea.

### BACKGROUND OF THE INVENTION

1. Field of the Invention

The invention generally relates to a disposable protective device. More particularly, the invention relates to a disposable protective device that is used for suction tube and is more hygienic.

2. Description of the Prior Art

When the respiratory tract is obstructed, the respiratory function would be adversely affected and blood oxygen level would become too low. Secretion and fluid in the respiratory tract may cause illnesses, such as pneumonia, bronchitis and asthma. Therefore, keeping the respiratory tract in an unobstructed condition is important. For a healthy person, keeping the respiratory tract in an unobstructed, clean condition is accomplished by mucociliary layer movement and cough. As dusts and particles are inhaled, they may stick to the mucociliary layer and transport onwards and upwards to the throat. Then, they are expelled by cough. However, if a person can not cough by himself or effectively (because of lung illnesses, endotracheal intubation, tracheostomy tube, drug effect and the fact that the viscosity level of secretion and fluid is affected by the temperature and humidity level of the air inhaled into the body), secretion, mucus and even blood would build up in the respiratory tract. Such build-up may be solved by suctioning. When a nurse or a medical person carries out suctioning, he or she must first put on a pair of sterile medical gloves and then place a suction tubeendotracheal tube or a tube inside the trachea. However, if the suction tube has no protective device medical persons may have contact with the secretion or bodily fluid of patients and then may contract diseases or spread the diseases.

From the above, we can see that the suction tube needs to be improved.

To eliminate the disadvantage, the inventor of the present invention has put a lot of effort into the subject and has successfully come up with the disposable protective device of the present invention.

### SUMMARY OF THE INVENTION

The object of the present invention is to provide a disposable protective device for suction tube to avoid the direct contact of bodily secretion and fluid and thus avoid the infection and spread of diseases.

To reach the object, the disposable protective device of the present invention is disclosed. The disposable protective device of the present invention comprises an encapsulating bag unit and a connective tubular unit. The encapsulating bag unit has a certain length and has a protruding circular portion which is located on the lower portion of the bag unit. The encapsulating bag unit has an interior space which may house a suction tube. The encapsulating bag unit has a first opening and a second opening at its two ends, with the diameter of the second opening larger than or equal to that of the first opening. The connective tubular unit has a first connector, a second connector and a third connector. The first connector is provided at the upper end of the connective tubular unit and may connect with the second opening. A cover may cover the first connector. The second connector is disposed near the center point of the connective tubular unit and may connect with a ventilator. The third connector is provided at the bottom part of the connective tubular unit and may connect with a tracheostomy tube or a tube inside the trachea.

### BRIEF DESCRIPTION OF THE DRAWINGS

The features and purposes of the disposable protective device for suction tube of the present invention would be better understood if the following is read with the accompanied drawings.

FIG. 1 is a side view illustrating the disposable protective device of the present invention.

FIG. 2 is a view of the disposable protective device of the present invention in an unconnected condition.

FIG. 3 is a perspective view illustrating the disposable protective device of the present invention connected with an oxygen supplying device.

FIG. 4 is a view illustrating the disposable protective device of the present invention in a fully connected condition.

FIG. 5 is a view illustrating the disposable protective device of the present invention in use.

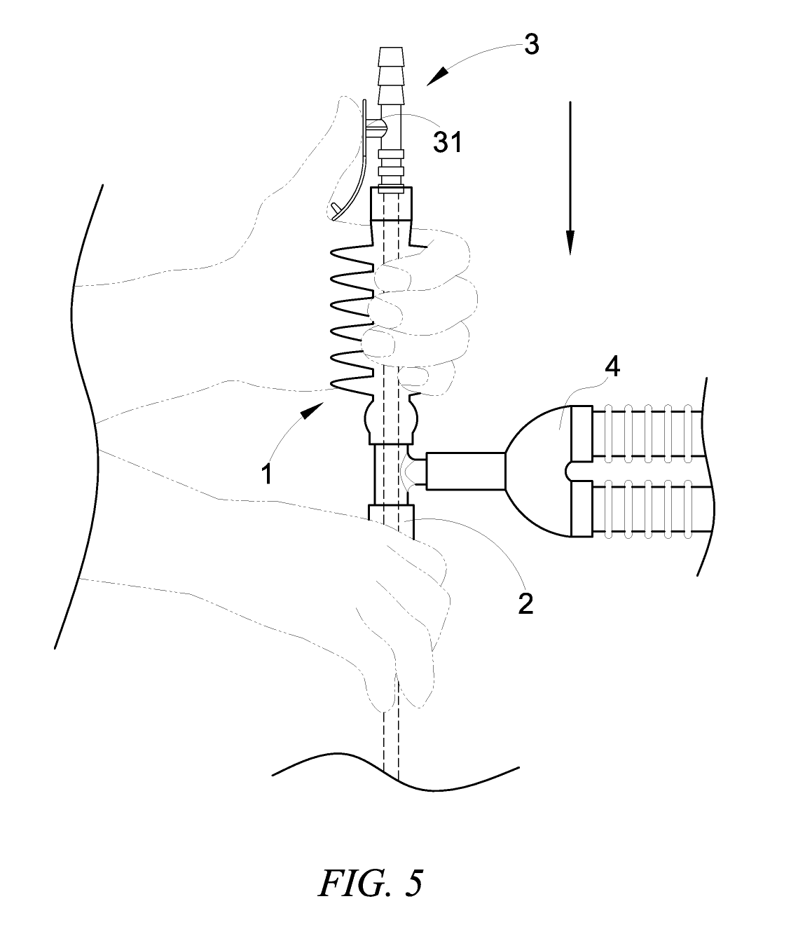

### DETAILED DESCRIPTION OF THE PREFERRED EMBODIMENT

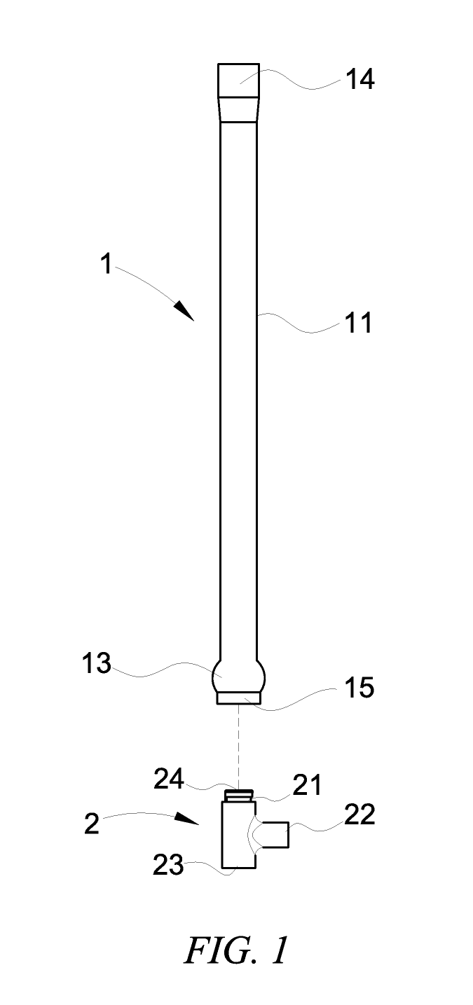

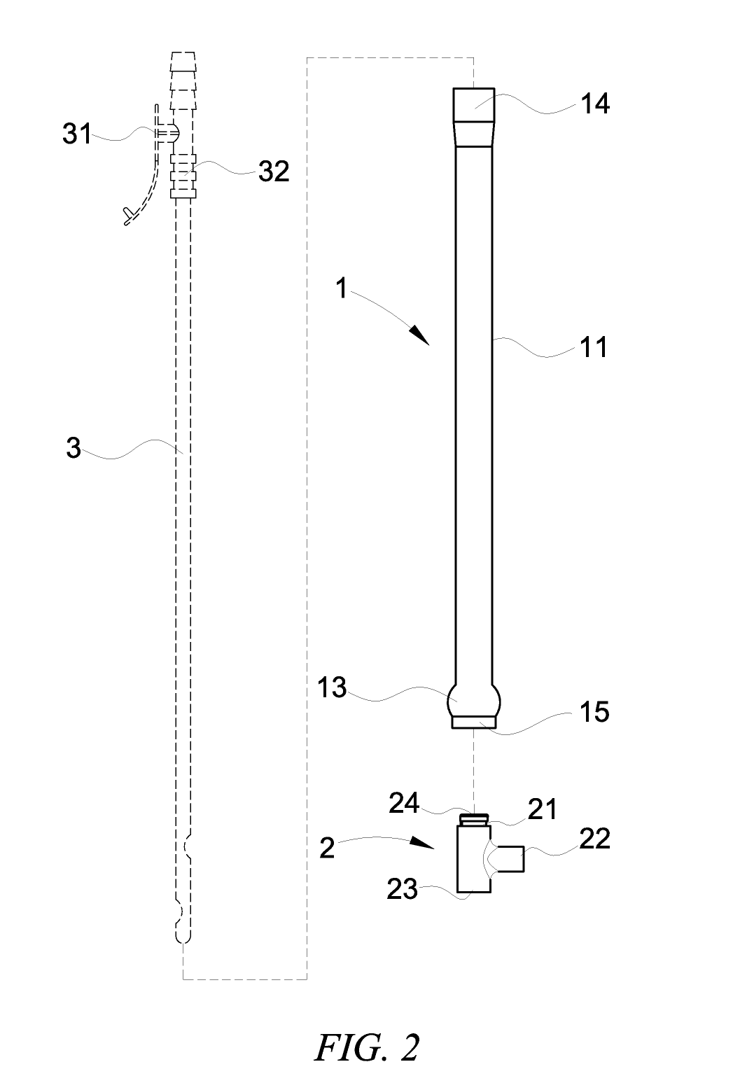

Please see FIGS. 1 and 2, which illustrate the disposable protective device of the present invention. The disposable protective device 1 of the present invention comprises an encapsulating bag unit 11 and a connective tubular unit 2. The encapsulating bag unit 11 has a certain length and has a protruding circular portion 13 which is located on the lower portion of the bag unit 11. The encapsulating bag unit 11 has an interior space 12 which may house a suction tube 3. The suction tube 3 may be put into and taken out of the interior space 12 easily. The encapsulating bag unit 1 has a first opening 14 and a second opening 15. The diameter of the second opening 15 is larger than or equal to that of the first opening 14. The connective tubular unit 2 has a first connector 21, a second connector 22 and a third connector 23. The first connector 21 may connect with the second opening 15. A cover 24 may cover the first connector 21. The second connector 22 is disposed near the center point of the connective tubular unit 2 and may connect with a ventilator 4 which is connected with an oxygen supplying device 5. The third connector 23 is provided at the bottom part of the connective tubular unit and may connect with a tracheostomy tube or a tube inside the trachea. A protective water-proof layer is provided on the surface of the bag unit 11. The first opening 14 may engage with a seat portion 32 of the suction tube 3. The protruding circular portion 13 may house the cover 24. The cover 24 may be opened and closed via the protruding circular portion 13.

In the medical suctioning practice, first, open the package of a suction tube 3. Then, put on a pair of sterile medical gloves. Then, connect the suction tube 3 with a drainage tube. Next, turn on a suction machine. Check whether the meter of the suction machine works normally and adjust its suction pressure. Then, insert the suction tube 3 into a tracheostomy tube or a tube inside the trachea and the depth of such insertion should be 10 to 12.5 cm. Next, press a vent control 31 for no more than 15 seconds to suck out secretion. Last, disconnect the suction tube 3 from the drainage tube and fold one of the gloves inside-out to wrap around the suction tube 3. Last, let distilled water running through the drainage tube and then resume the provision of oxygen.

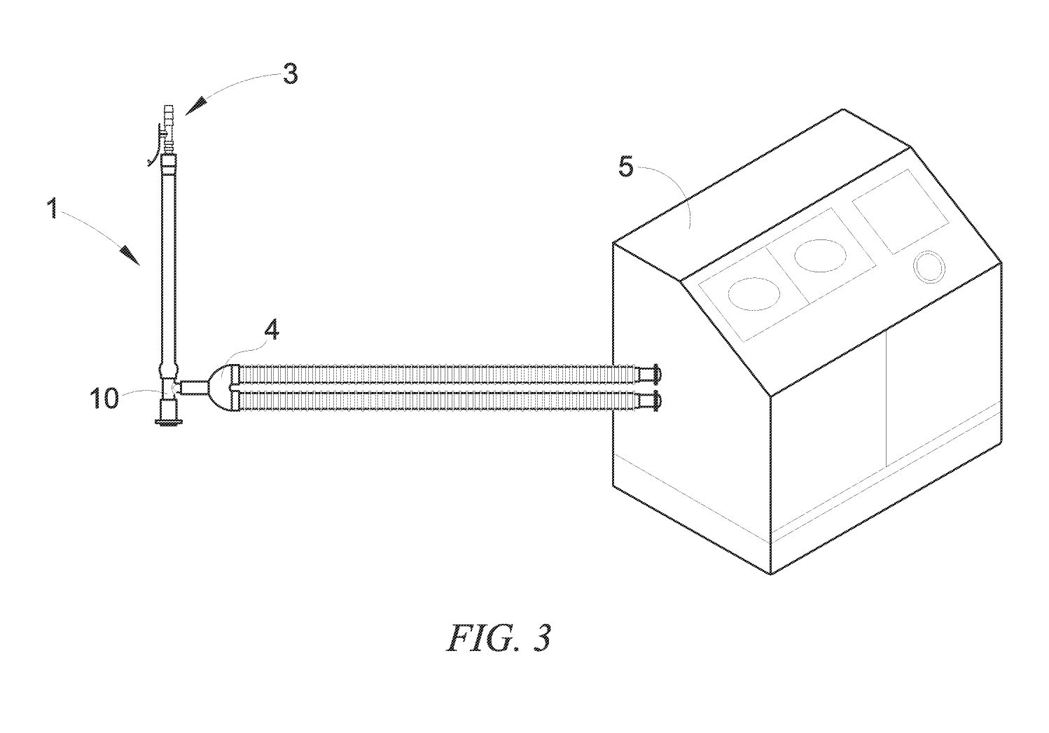

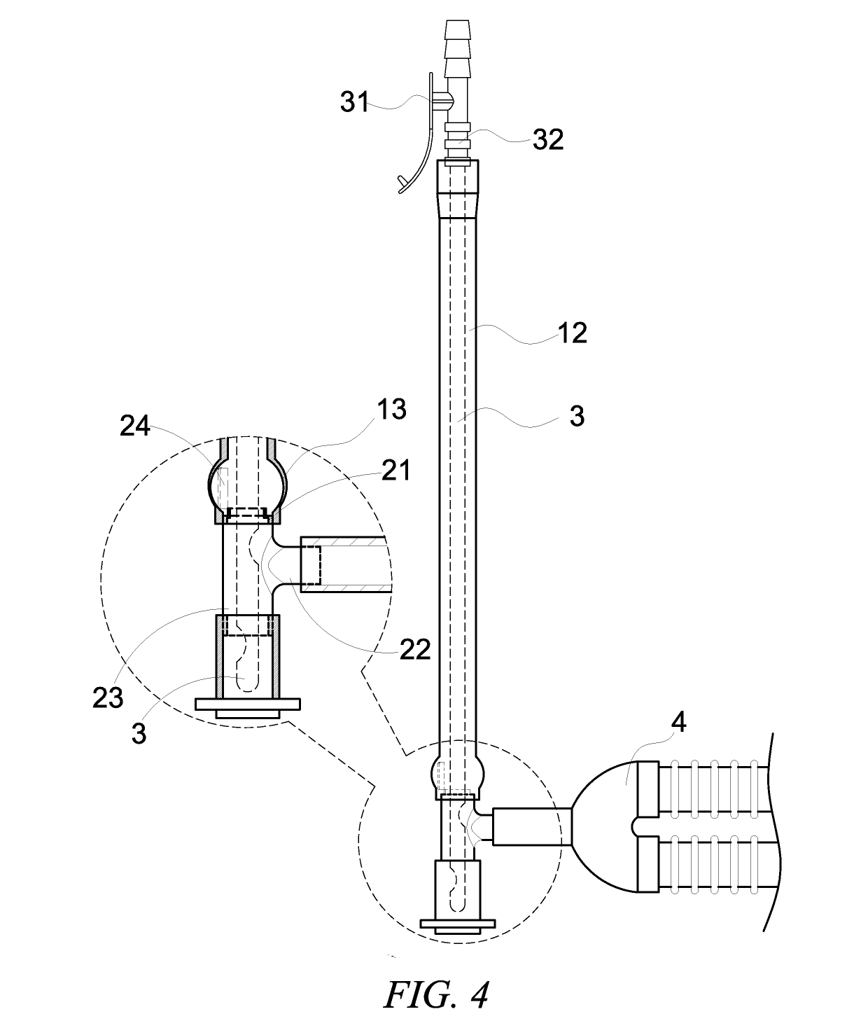

Please refer to FIGS. 3 and 4 for the use of the disposable protective device of the present invention. In use, first, connect the second opening 15 with the first connector 21. Then, place the suction tube 3 in the interior space 12 of the bag unit 11 and allow the first opening 14 to engage with a seat portion 32 of the suction tube 3 and allow the cover 24 to be opened via and around the protruding circular portion 13. Next, press a vent control 31 for no more than 15 seconds to suck out secretion from a tracheostomy tube or a tube inside the trachea. After suction is done, allow the cover 24 to be closed via and around the protruding circular portion 13 and then disconnect the second opening 15 from the first connector 21. Then, discard the suction tube 3 and the bag unit 1. Because suctioning is intrusive, the suction tube 3 should not be used again to avoid infection. In contrast to the suction tube of the prior art, the disposable protective device of the present invention may enable medical persons to avoid the contact of patients' secretion or bodily fluid so as to avoid infection.

Although a preferred embodiment of the present invention has been described in detail hereinabove, it should be understood that the preferred embodiment is to be regarded in an illustrative manner rather than a restrictive manner, and all variations and modifications of the basic inventive concepts herein taught still fall within the scope of the present invention.

From the above, we can see that the disposable protective device of the present invention meets the relevant patent requirements. It is hoped that the patent application will be approved.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A disposable protective device for suction tube, comprising:
an encapsulating bag unit, having a certain length and having a protruding circular portion which is located on the lower portion of the bag unit, wherein the encapsulating bag unit has an interior space which may house a suction tube, and the encapsulating bag unit has a first opening and a second opening at its two ends, with a diameter of the second opening larger than or equal to that of the first opening; and
a connective tubular unit, having a first connector, a second connector and a third connector, wherein the first connector is provided at the upper end of the connective tubular unit and may connect with the second opening, and that a cover may cover the first connector, the second connector is disposed near the center point of the connective tubular unit and may connect with a ventilator, and the third connector is provided at the bottom part of the connective tubular unit and may connect with a tracheostomy tube or a tube inside the trachea.

2. The disposable protective device as in claim 1, wherein a protective water-proof layer is provided on a surface of the encapsulating bag unit.

3. The disposable protective device as in claim 1, wherein the first opening may engage with a seat portion of the suction tube.

4. The disposable protective device as in claim 1, wherein the protruding circular portion may house the cover and the cover may be opened and closed via and around the protruding circular portion.
